(19) United States
(12) Patent Application Publication
(10) Pub. No.: US 2023/0225597 A1
(43) Pub. Date: Jul. 20, 2023

(54) ENDOSCOPIC IMAGING DEVICE AND ENDOSCOPE

(71) Applicant: JAPAN LIFELINE CO., LTD., Tokyo (JP)

(72) Inventor: Yuki KODAMA, Tokyo (JP)

(21) Appl. No.: 17/964,962

(22) Filed: Oct. 13, 2022

(30) Foreign Application Priority Data
Jan. 20, 2022 (JP) 2022-007122

(51) Int. Cl. A61B 1/012; A61B 1/005

(52) U.S. Cl. CPC A61B 1/012; A61B 1/005; A61B 1/0008

(57) ABSTRACT

A cholangioscope includes a catheter that is to be inserted into a body and has a curved shape, a camera shaft that can be inserted into the catheter and is formed in a curved shape, and a CMOS image sensor that is provided at a distal end portion of the camera shaft. The catheter further includes a forceps channel into which a medical instrument can be inserted, and a camera channel that is provided in an outer side portion of the catheter having the curved shape outer than the forceps channel and into which the camera shaft can be inserted. Curvature of the camera shaft is less than curvature of the catheter. The camera shaft is formed into a curved shape by using a shape memory alloy.

### TECHNICAL FIELD

The present invention relates to an endoscopic imaging device and the like that is included in an endoscope.

### BACKGROUND ART

Patent Literature 1 discloses an endoscopic imaging device (camera) with an imaging element provided at the distal end portion of a camera shaft, which can be separated from a catheter (hereinafter also referred to as a sheath) into which the camera shaft can be inserted. An endoscope includes the catheter equipped with the endoscopic imaging device, and the imaging element exposed at the distal end portion of the camera shaft inserted into the body together with the catheter images an image of a diagnostic site or a treatment site in the body. After use, the endoscopic imaging device can be detached from the sheath, which is typically used once, and can be reused with other sheaths of the same type within a predetermined number of times of use. By reusing an expensive endoscopic imaging device (or an imaging element), the cost per procedure can be significantly reduced.

### CITATION LIST

### Patent Literature

Patent Literature 1: WO 2021/191989

### SUMMARY OF INVENTION

### Technical Problem

In Patent Literature 1, an endoscopic imaging device inserted into a camera channel of a catheter may rotate in an unexpected direction within the camera channel.

The present invention has been made in view of such circumstances, and an object thereof is to provide an endoscopic imaging device and the like that can reduce rotation within a catheter. Alternatively, the present invention provides an endoscope and the like that can prevent misrecognition of the direction of an imaged image even when the endoscopic imaging device rotates within the catheter.

### Solution to Problem

To solve the above-mentioned problems, an endoscopic imaging device according to an aspect of the present invention includes a curved camera shaft that can be inserted into a curved catheter that is to be inserted into a body, and an imaging element provided at a distal end portion of the camera shaft.

In this aspect, a curved camera shaft is guided by a similarly curved catheter, which can reduce rotation of the camera shaft within the catheter.

Another aspect of the present invention is an endoscope. This endoscope includes a curved catheter that is to be inserted into a body, a curved camera shaft that can be inserted into the catheter, and an imaging element that is provided at a distal end portion of the camera shaft.

Still another aspect of the present invention is also an endoscope. This endoscope includes a catheter that is to be inserted into a body, a camera shaft that can be inserted into the catheter, an imaging element that is provided at a distal end portion of the camera shaft, and a direction indicator that is located at a distal end portion of the catheter and can be imaged by the imaging element.

In this aspect, even if the camera shaft rotates within the catheter, misrecognition of the direction of the image imaged by the imaging element can be prevented based on the direction indicator imaged by the imaging element.

Still another aspect of the present invention is also an endoscope. This endoscope includes a catheter that is to be inserted into a body, a camera shaft that can be inserted into the catheter, and an imaging element that is provided at a distal end portion of the camera shaft. In the catheter, a cross-sectional shape of at least a part of the camera channel into which the camera shaft can be inserted and a cross-sectional shape of at least a part of the camera shaft are non-circular and substantially similar.

In this aspect, the camera channel and camera shaft having substantially similar cross-sectional shapes can reduce rotation of the camera shaft within the camera channel.

### Advantageous Effects of Invention

According to the present invention, the rotation of the endoscopic imaging device within the catheter can be reduced. Alternatively, according to the present invention, misrecognition of the direction of the imaged image can be prevented even when the endoscopic imaging device is rotated within the catheter.

### BRIEF DESCRIPTION OF DRAWINGS

FIG. 1 illustrates an entire cholangioscope.

FIG. 2 is a side view of a distal end portion of a catheter.

FIG. 3 is a plan view of a distal end surface of the distal end portion of the catheter.

FIGS. 4A and 4B illustrate how a camera position switching unit switches a position of a camera head between a first position proximate to a proximal end and a second position proximate to a distal end.

FIG. 5 illustrates a camera removed from the cholangioscope.

FIGS. 6A and 6B illustrate a portion proximate to the distal end of the catheter and the camera in a cholangioscope according to a first embodiment.

FIGS. 7A and 7B illustrate a portion proximate to the distal end of the catheter and the camera in the cholangioscope according to the first embodiment.

FIGS. 8A to 8C illustrate details of a portion proximate to the distal end of the camera shaft before a curved shape is memorized in a shape memory alloy.

FIGS. 9A and 9B illustrate one cross-section of the catheter and the camera in a cholangioscope according to a second embodiment.

FIG. 10 illustrates a cholangioscope according to a third embodiment together with functional blocks implemented by a computer or the like outside the body connected to a connector provided at a proximal end portion of a camera.

FIGS. 11A and 11B illustrate an example of image processing by an image processing unit.

### DESCRIPTION OF EMBODIMENTS

Hereinafter, embodiments for carrying out the present invention will be described in detail with reference to the drawings. In the description or drawings, the same or equivalent constituent elements, members, and processing operations are denoted by the same reference numerals, and overlapping descriptions are omitted. The scales and shapes of the illustrated parts are set for convenience to facilitate the explanation and should not be construed as limiting unless otherwise specified. The embodiments are illustrative and do not limit the scope of the present invention in any way. Not all features or combinations of features described in the embodiments are essential to the present invention.

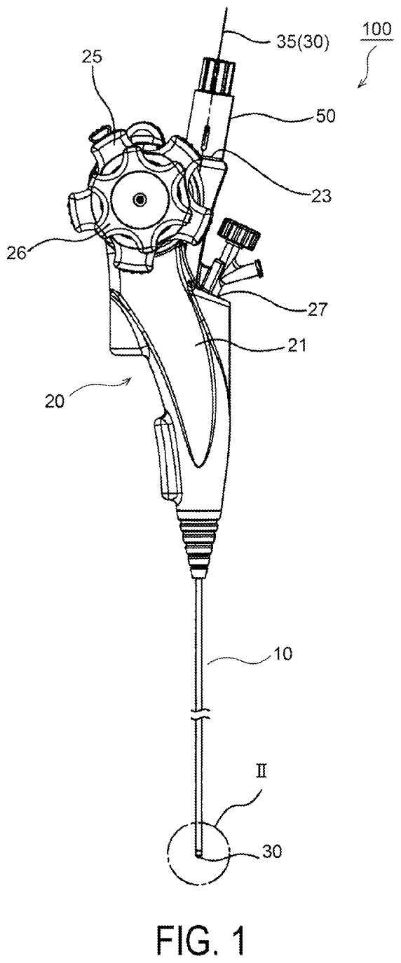

FIG. 1 illustrates an entire cholangioscope 100 to which an embodiment of the present invention is applied. The cholangioscope 100 is an endoscope that images the inside of the common bile duct and the pancreatic duct. The cholangioscope 100 includes a long tubular catheter 10 or sheath that is to be inserted into the body through the mouth or the like. At the distal end portion of the catheter 10 (lower end in FIG. 1), a camera head of a camera 30 as an endoscopic imaging device including an imaging element such as a CMOS image sensor is provided in an exposable state. The catheter 10 inserted into the duodenum through the mouth or the like is inserted into the common bile duct or the pancreatic duct through the papilla of Vater facing the duodenum. In this state, the camera head exposed at the distal end portion of the catheter 10 images an image such as a moving image or a still image of the interior of the common bile duct and the pancreatic duct for diagnosis or treatment.

By inserting the catheter 10 of the cholangioscope 100 into the forceps channel or the like of a duodenoscope (not illustrated) inserted into the duodenum from the mouth or the like, the cholangioscope 100 can be reliably guided to the duodenum, and the cholangioscope 100 can be reliably inserted into the narrow papilla of Vater while confirming the images obtained from the duodenoscope. Before inserting the catheter 10 into the papilla of Vater, a guide wire having a smaller diameter than that of the catheter 10 may be inserted into the papilla of Vater through the wire lumen of the duodenoscope, and the papilla of Vater may be expanded in advance by a balloon catheter guided through the guide wire.

A handle portion 20 for operating the cholangioscope 100 is provided in a portion proximate to the proximal end (upper end in FIG. 1) of the cholangioscope 100 or the catheter 10 or a portion of the cholangioscope 100 or the catheter 10 outside the body. The handle portion 20 has a grip portion 21 that is gripped by an operator such as a physician, and two rotary operating portions 25 and 26 that allow the operator to bend at least the distal end portion of the catheter 10 in any direction to adjust the position. Because the camera head of the camera 30 is disposed at the distal end portion of the catheter 10 as described above, the imaging portion and the imaging range of the camera 30 can be freely changed by rotating the two rotary operating portions 25 and 26.

As will be described below, inside the catheter 10 or sheath, there are provided a camera channel 13 into which a small-diameter camera shaft with a camera head provided at the distal end portion thereof is inserted, and a forceps channel 17, as a large-diameter instrument channel, into which various medical instruments for examination or treatment of the inside of the common bile duct or pancreatic duct are inserted. The handle portion 20 is provided with a camera port 23 communicating with the camera channel 13 of the catheter 10 and a forceps port 27 communicating with the forceps channel 17 of the catheter 10. That is, the small-diameter camera shaft with the camera head provided at distal end portion thereof is inserted from the camera port 23 into the sheath 10 (camera channel 13), and various medical instruments are inserted from the forceps port 27 into the sheath 10 (forceps channel 17).

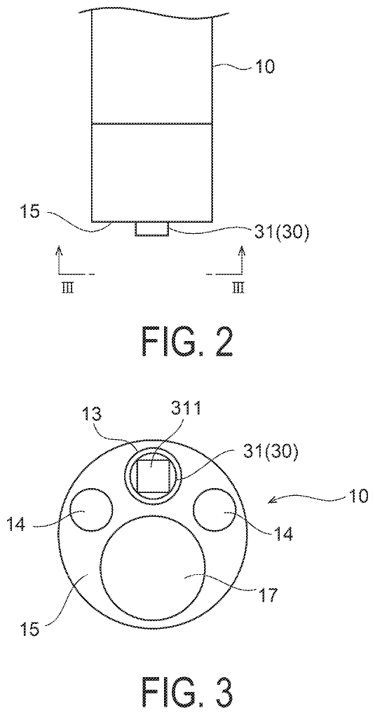

FIG. 2 is a side view of the distal end portion of the catheter 10, and FIG. 3 is a plan view of a distal end surface 15 of the distal end portion of the catheter 10. As illustrated in FIG. 3, the cross-section of the catheter 10 is substantially circular and is provided with the camera channel 13, the forceps channel 17 and two air/water channels 14, each having a substantially circular cross-section. The camera channel 13, the forceps channel 17, and the air/water channels 14 communicate with the outside of the body through the sheath 10. As described above, the camera channel 13 communicates with the camera port 23, the forceps channel 17 communicates with the forceps port 27, and the air/water channels 14 communicate with the air/water ports provided in the forceps port 27.

A small-diameter long camera shaft with the camera head 31 provided at its distal end portion is inserted into the camera channel 13. The camera head 31 constitutes the distal end portion of the camera 30 as an endoscopic imaging device and includes a CMOS image sensor 311 as an imaging element. The camera head 31 or the CMOS image sensor 311 is connected to a power source, a computer, and the like outside the body by lead wires passing through the camera shaft and a camera cable 35 extending from a camera connector 50 of FIG. 1 to the outside of the body. Through this lead wire, power is supplied from the power source outside the body to the CMOS image sensor 311, control signals are transmitted from a control device such as a computer outside the body to the CMOS image sensor 311, and image signals imaged by the CMOS image sensor 311 are transmitted to a computer, a monitor, and the like outside the body. It should be noted that transmission of control signals and image signals between a computer or the like outside the body and the CMOS image sensor 311 may be performed wirelessly by Bluetooth (trade name) or the like without using lead wires.

While a light receiving surface of the CMOS image sensor 311 is rectangular (typically square), the distal end surface of the camera head 31 (which is also the distal end surface of the camera 30 or the camera shaft) circumscribing the light receiving surface is circular. A plurality of optical fibers (not illustrated) are provided in a region between the circular outer periphery of the camera head 31 and the rectangular outer periphery of the CMOS image sensor 311, and thus the optical fibers surround the outer periphery of the CMOS image sensor 311. These optical fibers extend out of the camera port 23 of FIG. 1 through the camera shaft in the same manner as the lead wires connected to the CMOS image sensor 311 and are connected to a light source outside the body. The light from this light source is emitted from the distal end surface of the optical fiber and illuminates the imaging range of the CMOS image sensor 311, and thus a clear image of the diagnostic site or the treatment site can be obtained.

Various medical instruments with treatment tools such as forceps provided at their distal end portions are inserted into the forceps channel 17. The air/water channels 14 supply air and water from air/water ports provided in the forceps port 27 to the diagnostic site or treatment site. The small-diameter camera channel 13 and the large-diameter forceps channel 17 are aligned, and thus their centers are disposed on the same straight line (on the vertical line in FIG. 3) as the center of the distal end surface 15 of the catheter 10. In FIG. 3, the camera channel 13 and the forceps channel 17 are aligned vertically, and two air/water channels 14 are symmetrically disposed in the upper left and right regions (proximate to the small-diameter camera channel 13).

In FIG. 3, the diameter of the catheter 10 or sheath is from 1.1 mm to 7.0 mm (for example, 3.6 mm), the diameter of the camera channel 13 is from 0.7 mm to 3.0 mm (for example, 1.1 mm), and the diameter of the forceps channel 17 is from 0.3 mm to 3.0 mm (for example, 2.0 mm). The diameter of the camera shaft and camera head 31 inserted into the camera channel 13 is slightly smaller than the diameter of the camera channel 13 and is from 0.6 mm to 2.9 mm (for example, 1.0 mm). If the forceps channel 17 has a diameter of 2.0 mm or more, it can be used with typical forceps or medical instruments having a diameter of 1.8 mm. On the other hand, the diameter of the camera channel 13 can be reduced according to the miniaturization of the CMOS image sensor 311. Thus, according to the present embodiment, because the diameter of the camera channel 13 can be minimized (for example, 1.1 mm or less) while maintaining the minimum diameter (for example, 2.0 mm or more) of the forceps channel 17 compatible with typical forceps or medical instruments, it is possible to implement a sufficiently thin catheter 10 that can easily pass through the narrow papilla of Vater.

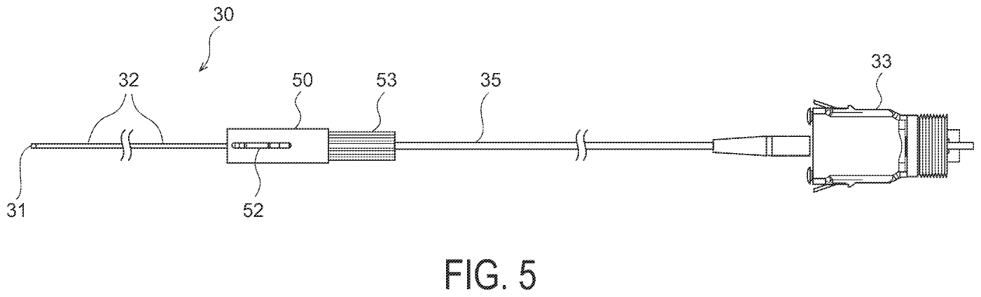

As illustrated in FIG. 2, the camera head 31 constituting the distal end portion of the camera 30 as an endoscopic imaging device is provided in such a manner that the camera head 31 can further protrude and expose from the distal end surface 15 of the catheter 10 in a direction from the proximal end to the distal end of the catheter 10. As described above, the position at which the camera head 31 or the CMOS image sensor 311 further protrudes from the distal end surface 15 of the catheter 10 in the direction from the proximal end to the distal end of the catheter 10 will be also referred to as a second position. On the other hand, the camera head 31 or the CMOS image sensor 311 can also take a position (hereinafter also referred to as a first position) retracted from the distal end surface 15 of the catheter 10 in a direction toward the proximal end of the catheter 10. Switching between the first position and the second position can be performed by the operator using a camera position switching unit 53 (FIG. 5) provided side by side with the camera connector 50.

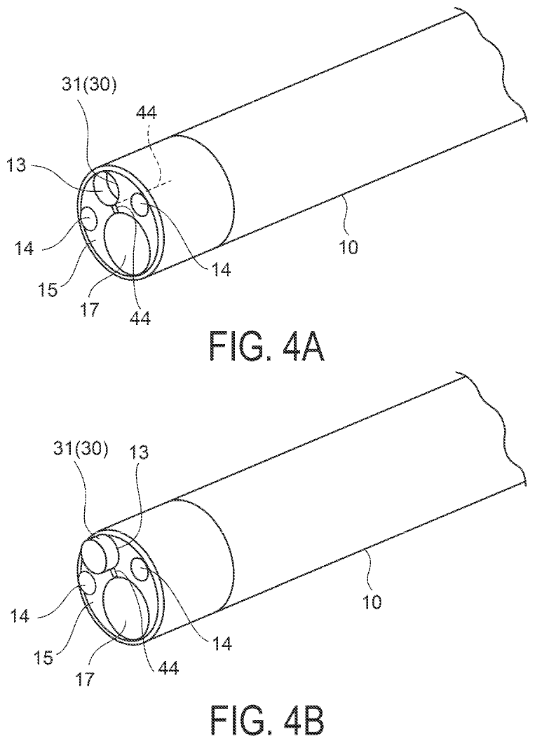

FIGS. 4A and 4B illustrate how the camera position switching unit 53 switches the camera head 31 between a first position proximate to the proximal end and a second position proximate to the distal end. In FIG. 4A, the camera head 31 is at a first position retracted from the distal end surface 15 of the catheter 10 toward the proximal end of the catheter 10, and in FIG. 4B, the camera head 31 is at a second position projected from the distal end surface 15 of the catheter 10 in the direction from the proximal end to the distal end of the catheter 10. The retraction distance between the distal end surface 15 of the catheter 10 and the first position is, for example, from 1.5 mm to 20 mm, and the protruding distance between the distal end surface 15 of the catheter 10 and the second position is, for example, from 0.5 mm to 80 mm.

When the CMOS image sensor 311 of the camera head 31 images an image of a diagnostic site or a treatment site, the second position in FIG. 4B is taken. When the distal end portion of the catheter 10 is inserted into the common bile duct or pancreatic duct as an imaging portion, the first position of FIG. 4A in which the camera head 31 is housed in the catheter 10 or sheath is taken, and thus the camera head 31 does not come into contact with the inner wall of the duodenoscope that guides the catheter 10 to the duodenum, the inner wall of the papilla of Vater constricted by the sphincter of Oddi (or the sphincter of the biliary pancreatic ampulla), the common bile duct, or the pancreatic duct. The position switching mechanism of the camera head 31 by such a camera position switching unit 53 is disclosed in International Patent Application No. PCT/JP2020/012793 (WO 2021/191989) filed on Mar. 23, 2020, and the entire contents of which are incorporated herein by reference.

The camera 30 as an endoscopic imaging device is detachable from the catheter 10 or the cholangioscope 100. FIG. 5 illustrates the camera 30 detached from the cholangioscope 100. The camera 30 includes the small-diameter (from 0.6 mm to 2.9 mm) and long tubular camera shaft 32 with the camera head 31 provided at its distal end portion (the left end portion in FIG. 5), the camera connector 50 connected to the proximal end portion (the right end portion in FIG. 5) of the camera shaft 32, and the camera cable 35 electrically connecting the camera connector 50 and the connector 33 provided at the proximal end portion of the camera 30. As described above, the lead wire connected to the CMOS image sensor 311 of the camera head 31 at the distal end portion of the camera 30 passes through the camera shaft 32 and the camera cable 35 and is connected to a power source, a computer, a monitor, and the like outside the body via the connector 33.

As illustrated in FIG. 1, the camera connector 50 is attachable/detachable to/from the camera port 23 of the cholangioscope 100. However, as disclosed in International Patent Application No. PCT/JP2020/012793, an attachment restricting mechanism is provided to restrict the camera shaft 32 from being inserted into the catheter 10 (the camera channel 13) in a state (FIG. 4B) in which the camera head 31 can protrude from the distal end surface 15 of the catheter 10. As a result, the camera 30 (camera connector 50) can be attached to the cholangioscope 100 (camera port 23) only when the camera head 31 is in a position where the camera head 31 does not protrude from the distal end surface 15 of the catheter 10, that is, when the camera head 31 is at the first position (FIG. 4A).

Switching between the first position and the second position of the camera head 31 is performed by rotating the camera position switching unit 53 provided side by side with the camera connector 50. When the camera position switching unit 53 is rotated in a first direction, the camera head 31 moves to the first position (FIG. 4A) and a slide member 52 provided on the camera connector 50 moves in a direction toward the proximal end (the right side in FIG. 5). Further, when the camera position switching unit 53 is rotated in a second direction opposite to the first direction, the camera head 31 moves toward the second position (FIG. 4B), and the slide member 52 provided on the camera connector 50 moves in a direction toward the distal end (the left side in FIG. 5). Thus, it can be said that the camera 30 (camera connector 50) can be attached to the cholangioscope 100 (camera port 23) only when the slide member 52 is in a position proximate to the proximal end.

As described above, the camera head 31 attached to the cholangioscope 100 at the first position (FIG. 4A) is moved to the second position (FIG. 4B) with the rotation of the camera position switching unit 53 in the second direction after reaching the imaging portion in the body to perform imaging. After completion of imaging, the camera head 31 is returned to the first position by the rotation of the camera position switching unit 53 in the first direction. By removing the catheter 10 from the body in this state, the camera head 31 retracted further than the distal end surface 15 of the catheter 10 in the direction toward the proximal end of the catheter 10 can be prevented from coming into contact with the body tissue or the inner wall of the main endoscope such as the duodenoscope. Furthermore, after use, the camera 30 is detached from the sheath 10, which is typically used once (specifically, the camera 30 is detached from the camera port 23 of the cholangioscope 100). This camera 30 can be reused for other sheaths 10 or cholangioscopes 100 of the same type within a range not exceeding a predetermined number of uses (for example, 10 times). By reusing the expensive camera 30 (or CMOS image sensor 311), the cost per procedure can be greatly reduced.

As described above, in the cholangioscope 100 in which the camera 30 is detachable, there is a possibility that the camera shaft 32 inserted into the camera channel 13 of the catheter 10 rotates within the camera channel 13. In the following embodiments, the camera 30 and the cholangioscope 100 that can reduce the rotation within the catheter 10, and the cholangioscope 100 that can prevent misrecognition of the direction of the imaged image even when the camera 30 rotates within the catheter 10 are to be disclosed. A plurality of embodiments will be individually described below, but part or all of the embodiments can be combined freely unless there is a particular problem.

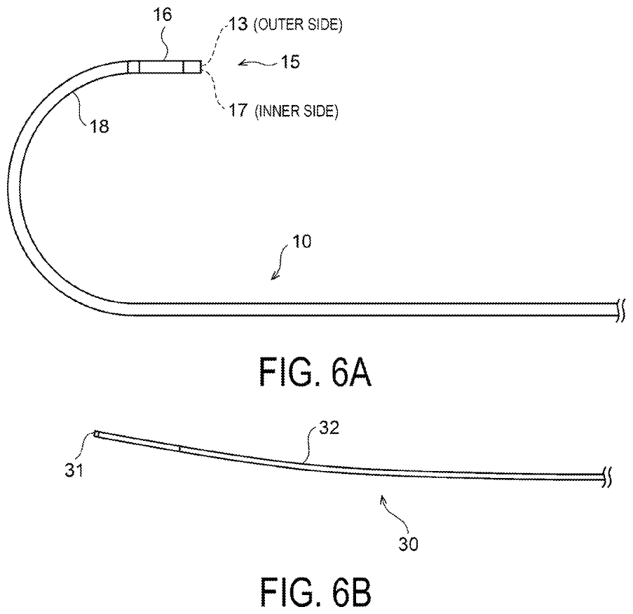

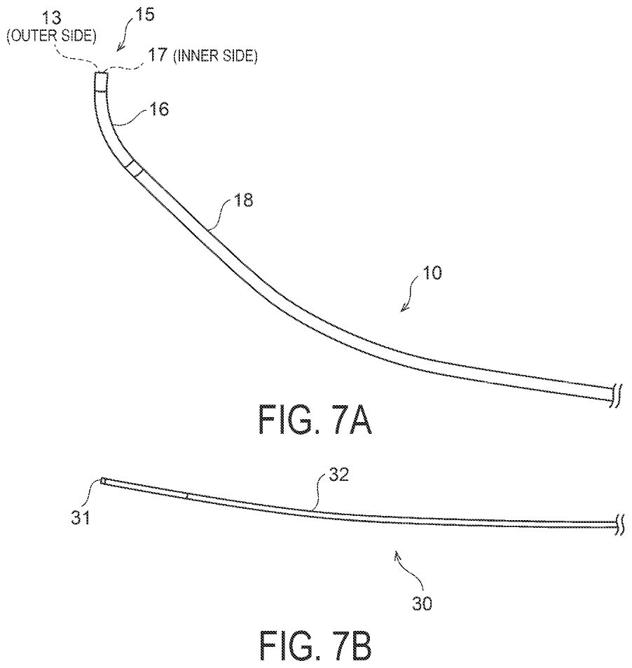

FIGS. 6 and 7 illustrate portions proximate to the distal end of the catheter 10 (FIGS. 6A and 7A) and portions proximate to the distal end of the camera 30 (FIGS. 6B and 7B) in the cholangioscope 100 according to the first embodiment. The catheter 10 is formed in a curved shape, and thus the catheter 10 can be easily inserted into the common bile duct or pancreatic duct through the papilla of Vater on the side wall of the duodenum. As described above, the distal end portion 16 of the catheter 10 can be bent in any direction by the rotary operating portions 25 and 26. However, the catheter 10 is formed in a curved shape while including also the catheter body 18 proximate to the proximal end of the catheter 10 from the distal end portion 16. The catheter 10 of FIG. 6A has a relatively large curvature, and the distal end surface 15 faces in a direction of the proximal end of the catheter 10 (right side in FIG. 6). The catheter 10 of FIG. 7A has a relatively small curvature, and the distal end surface 15 does not face in the direction of the proximal end of the catheter 10.

Here, the camera channel 13 into which the camera shaft 32 is inserted is provided in an outer side portion of the curved catheter 10, and the forceps channel 17 into which various medical instruments for examination or treatment of the inside of the common bile duct or pancreatic duct are inserted is provided in an inner side portion of the curved catheter 10. In the curved catheter 10 and the curved camera shaft 32, which will be described below, “outer side portion” refers to a portion that protrudes in a convex shape and “inner side” refers to a portion that is depressed in a concave shape. In FIG. 3 illustrating the camera channel 13 and the forceps channel 17, the upper side corresponds to the outer side portion of the curved catheter 10 and the lower side corresponds to the inside portion of the curved catheter 10. Thus, the camera channel 13 is provided in the outer side portion of the curved catheter 10 outer than the forceps channel 17. For this reason, the curvature of the camera channel 13 is slightly less than the curvature of the forceps channel 17, and the camera shaft 32 can be easily inserted into the camera channel 13.

Similarly to a portion proximate to the distal end of the catheter 10, a portion proximate to the distal end of the camera 30 or the camera shaft 32 is also formed in a curved shape. When the camera shaft 32 is inserted into the camera channel 13, the curved camera shaft 32 is guided by the camera channel 13, which is also curved, and thus the rotation of the camera shaft 32 within the camera channel 13 can be reduced. As described above, according to the present embodiment, the detachable camera 30 can be inserted into the catheter 10 or the cholangioscope 100 in a desired direction. Even if the camera shaft 32 does not bend greatly like the catheter 10 or the camera channel 13, and if the camera shaft 32 bends enough to follow the curvature of the camera channel 13, the camera shaft 32 can maintain a substantially constant direction within the camera channel 13. Thus, the curvature of the camera shaft 32 may be smaller than the curvature of the catheter 10 or the camera channel 13.

Here, the curvature is the reciprocal of the radius (radius of curvature) of the curved portion. Although the curvature (and radius of curvature) can vary at each of the portions of the catheter 10 and each of the portions of the camera shaft 32, when the curvature of each of the curved portions of the catheter 10 and the curvature of the corresponding one of curved portions of the camera shaft 32, which are coming at the same position when the camera shaft 32 is inserted into the camera channel 13 of the catheter 10, are compared, it is preferable that the former be generally or on average greater than the latter. Alternatively, it is preferable that the maximum curvature of the catheter 10 is greater than the maximum curvature of the camera shaft 32. Moreover, when comparing the curvatures of each curved portion of the catheter 10 and the camera shaft 32, it is preferable to compare the curvatures of the respective central shafts. Alternatively, the inner (innermost) curvatures of the catheter 10 and the camera shaft 32 may be compared, or the outer (outermost) curvatures of the catheter 10 and camera shaft 32 may be compared.

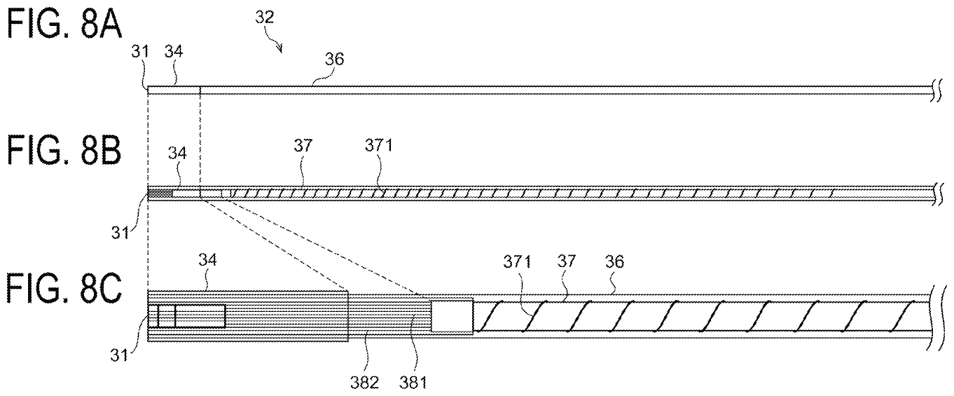

The camera shaft 32 is formed in a curved shape by using metal. Shape memory alloys such as NiTi (nickel titanium) alloys are suitable as metals. FIG. 8 illustrates details of a portion proximate to the distal end of the camera shaft 32 before the curved shape is memorized in metal such as a shape memory alloy. FIG. 8A illustrates the appearance of the camera shaft 32, FIG. 8B is a transparent view of the cover and the like on the surface of the camera shaft 32 in FIG. 8A, and FIG. 8C is an enlarged view of a portion proximate to the distal end of FIG. 8B. As illustrated in FIG. 8A, a cylindrical distal end cover 34 is provided at the distal end portion of the camera shaft 32 (the left end in FIG. 8) to cover and protect the outer periphery or side surface of the camera head 31 or the CMOS image sensor 311. The distal end cover 34 is made of hard resin or the like, and has a length of about 5 mm in the axial direction (the horizontal direction in FIG. 8) of the distal end cover 34.

A cylindrical shaft cover 36 that covers the outer periphery of the shape memory alloy tube 37 and the like, which will be described below, is provided on a portion extending from the distal end portion of the camera shaft 32 toward the proximal end (the right side in FIG. 8) of the camera shaft 32 so as to continue from the distal end cover 34. At least a portion proximate to the distal end of the shaft cover 36 is located inside the distal end portion 16 of the catheter 10 (the camera channel 13) in FIGS. 6A and 7A. By forming the shaft cover 36 with a resin or the like that is softer than the hard distal end cover 34, the camera shaft 32 (the shaft cover 36) can be flexibly bent following the distal end portion 16 of the catheter 10, which can be bent in any direction by the rotary operating portions 25 and 26.

The shape memory alloy tube 37 as a tubular or cylindrical metal tube along the camera shaft 32 is provided on the inner periphery covered by the shaft cover 36. The shape memory alloy tube 37 is a long circular tube made of a shape memory alloy such as a NiTi alloy and memorizes a desired curved shape of the camera shaft 32 as illustrated in FIGS. 6B and 7B by shape memory processing by temperature or magnetic field. The shape memory alloy tube 37 is not provided at the distal end portion (not intended to bend) of the camera shaft 32 where the hard distal end cover 34 is provided. A spiral cut 371 is provided in a portion proximate to the distal end of the shape memory alloy tube 37 by laser processing or the like. As a result, when the rotary operating portions 25 and 26 cause the distal end portion 16 (FIGS. 6A and 7A) of the catheter 10 to bend, the shape memory alloy tube 37 can also bend flexibly to follow the distal end portion 16. The pitch of the spiral cut 371 is reduced gradually or in a stepwise manner as in a direction from a proximal end of the shape memory alloy tube 37 to a distal end of the shape memory alloy tube 37 at least in a part of the shape memory alloy tube 37, and the closer to the distal end of the shape memory alloy tube 37 where the degree of bending caused by the rotary operating portions 25 and 26 is large, the easier the shape memory alloy tube 37 bends. On the other hand, the spiral cut 371 is not provided in a portion proximate to the proximal end of the shape memory alloy tube 37 where the degree of bending by the rotary operating portions 25 and 26 is very small (or absolutely zero). For example, the spiral cut 371 is formed in a range of about 25 mm from the distal end of the shape memory alloy tube 37 and is not formed in a portion extending therefrom to the proximal end of the shape memory alloy tube 37.

As illustrated in FIG. 8C, a plurality of lead wires 381 connected to the camera head 31 or the CMOS image sensor 311 and a plurality of optical fibers 382 disposed around the CMOS image sensor 311 pass through the distal end cover 34, the shaft cover 36, and the shape memory alloy tube 37 to extend to the outside of the camera port 23 in FIG. 1.

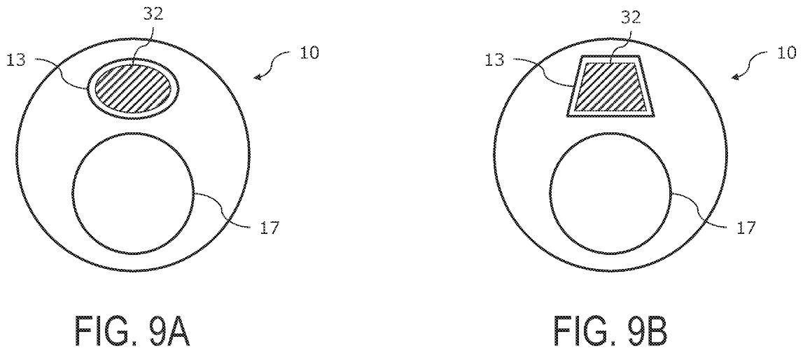

FIG. 9 illustrates one cross-section of a catheter 10 and a camera 30 (a camera shaft 32) in a cholangioscope 100 according to a second embodiment. The illustration of the air/water channels 14 in FIG. 3 illustrating the distal end surface of the catheter 10 and the camera 30 is omitted. In the present embodiment, a cross-sectional shape of at least a part of the camera channel 13 of the catheter 10 and a cross-sectional shape of at least a part of the camera shaft 32 of the camera 30 are non-circular and substantially similar.

In the example of FIG. 9A, a cross-sectional shape of at least a part of the camera channel 13 and a cross-sectional shape of at least a part of the camera shaft 32 are substantially similar elliptical. In the example of FIG. 9B, a cross-sectional shape of at least a part of the camera channel 13 and a cross-sectional shape of at least a part of the camera shaft 32 are substantially similar polygonal (specifically, trapezoidal, for example). A guide groove extending in the length direction may be provided on one of the inner periphery of the camera channel 13 or the outer periphery of the camera shaft 32, and a protruding portion that fits in the guide groove so as to be slidable or insertable in the length direction may be provided on the other of the inner periphery of the camera channel 13 or the outer periphery of the camera shaft 32 to achieve substantially similar shapes. The camera channel 13 and the camera shaft 32 having substantially similar cross-sectional shapes can reduce the rotation of the camera shaft 32 within the camera channel 13, thus allowing the detachable camera 30 to be inserted into the catheter 10 or the cholangioscope 100 in a desired direction.

Note that the substantially similar shape does not have to be a mathematically exact similar shape, as long as the shape is similar enough to effectively restrict the rotation of the camera shaft 32 within the camera channel 13. For example, even if the camera shaft 32 with a rectangular cross-section is inserted into the camera channel 13 with an elliptical cross-section, the rotation of the camera shaft 32 within the camera channel 13 can be restricted, and thus the requirements for substantially similar shapes in this specification are satisfied. The non-circular cross-section as illustrated in FIGS. 9A and 9B need not be formed along the entire length of the camera channel 13 and/or the camera shaft 32 but may be formed in at least one location along the length. When the cross-section of the camera channel 13 is formed in an elliptical or a polygonal shape over its entire length, the cross-section of the camera shaft 32 is a substantially circular shape included in the elliptical shape or the polygonal shape over its entire length. However, the rotation of the camera shaft 32 within the camera channel 13 may be restricted by attaching a rotation restricting portion having a shape substantially similar to the elliptical or polygonal shape to the outer circumference of at least one portion of the camera shaft 32 in the length direction.

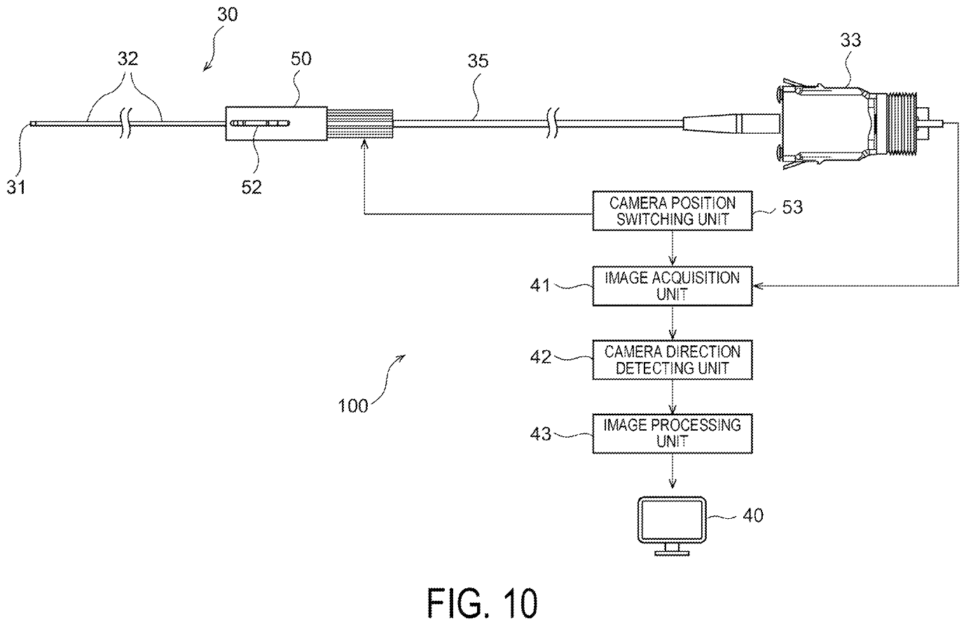

FIG. 10 illustrates a cholangioscope 100 according to a third embodiment together with functional blocks implemented by a computer or the like outside the body connected to a connector 33 provided at a proximal end portion of a camera 30. An image acquisition unit 41 acquires an image imaged by a CMOS image sensor 311 in a camera head 31 and transmitted through lead wires passing through a camera shaft 32 and a camera cable 35 from the connector 33. A camera direction detecting unit 42 detects a direction of the CMOS image sensor 311 relative to a catheter 10 (not illustrated in FIG. 10, see FIG. 3) based on a direction indicator to be described below included in the image acquired by the image acquisition unit 41. An image processing unit 43 processes the image acquired by the image acquisition unit 41 according to the direction of the CMOS image sensor 311 detected by the camera direction detecting unit 42 and displays the processed image on a monitor 40 or a display device.

The direction indicator indicating the direction of the image acquired by the image acquisition unit 41 relative to the catheter 10 is located at a distal end portion of the catheter 10 and is imaged by the CMOS image sensor 311 before or during the procedure using the cholangioscope 100. FIG. 4 illustrates an example of the direction indicator 44. The direction indicator 44 is a linear mark extending in the length direction of the catheter 10 on the portion of the inner wall of the distal end portion of the camera channel 13 that is closest to the forceps channel 17. As illustrated in FIG. 4, the linear direction indicator 44 may extend from the lower end of the camera channel 13 to the upper end of the forceps channel 17 on the distal end surface 15 of the catheter 10.

The CMOS image sensor 311 positioned at the first position in FIG. 4A by the camera position switching unit 53 can image the linear direction indicator 44 provided on the camera channel 13 and/or the forceps channel 17 or at the distal end portion of the catheter 10. Then, the camera direction detecting unit 42 can detect the direction of the CMOS image sensor 311 relative to the catheter 10, the camera channel 13 and the forceps channel 17 based on the direction indicator 44 included in the image acquired by the image acquisition unit 41. In this manner, based on the direction indicator 44 imaged by the CMOS image sensor 311, misrecognition of the direction of the image imaged by the CMOS image sensor 311 can be prevented even when the camera shaft 32 rotates within the catheter 10. Note that the direction indicator 44 is not limited to the mark as described above and may be any object or structure that suggests the direction of the CMOS image sensor 311. For example, the contour of the distal end portion of the camera channel 13, the contour of the distal end portion of the forceps channel 17, the contour of the distal end surface 15 of the catheter 10, the medical instrument protruding from the distal end portion of the forceps channel 17, or the like may be imaged by the CMOS image sensor 311 as the direction indicator 44.

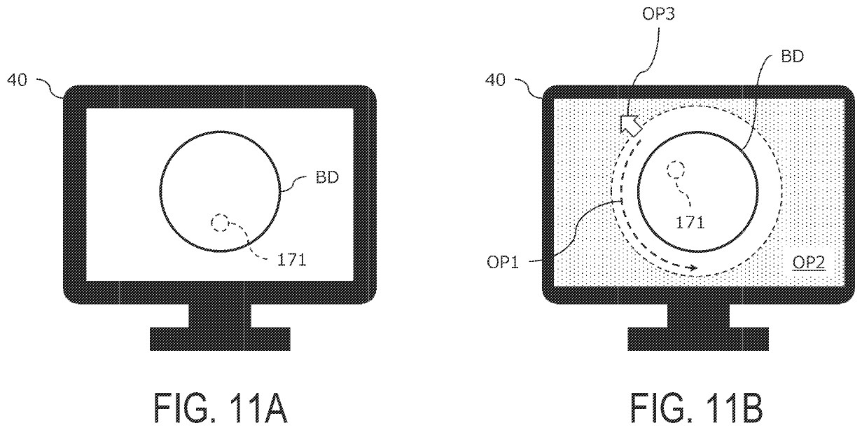

The image processing unit 43 processes the image acquired by the image acquisition unit 41 according to the direction of the CMOS image sensor 311 detected by the camera direction detecting unit 42. FIG. 11 illustrates an example of image processing by the image processing unit 43. FIG. 11A illustrates an example of an image imaged by the CMOS image sensor 311 and displayed on the monitor 40 in an ideal case where the CMOS image sensor 311 faces the desired direction relative to the forceps channel 17 and the like. A substantially circular common bile duct (BD or Bile Duct) is displayed in the center of the screen, and a medical instrument 171 led out from the forceps channel 17 emerges exactly below (vertically below) the center of the screen. In contrast, in FIG. 11B, the camera shaft 32 rotates within the catheter 10, which results in a rotation of the emerging position of the medical instrument 171 from a position illustrated in FIG. 11A.

As indicated by the first option OP1, the image processing unit 43 rotates the image imaged by the CMOS image sensor 311, and thus the direction of the CMOS image sensor 311 detected by the camera direction detecting unit 42 matches the desired direction in FIG. 11A. As a result, the monitor 40 displays an image similar to that illustrated in FIG. 11A. At this time, as indicated by the second option OP2, the image processing unit 43 may cut out the image that is a target of rotation by the first option OP1, over a predetermined diameter range around the center of the rotation. When the image is rotated, a part of the originally rectangular image protrudes outside the screen and the outline of the image is lost. However, this influence can be eliminated by cutting the image in a circular shape. The predetermined diameter range cut out by the image processing unit 43 preferably includes at least the common bile duct BD as an imaging target.

As indicated by the third option OP3, the image processing unit 43 displays the direction of the direction indicator 44 imaged by the CMOS image sensor 311 (the direction of the forceps channel 17 or the direction in which the medical instrument 171 emerges in the examples of FIGS. 4 and 11) on the image imaged by the CMOS image sensor 311. In the example of FIG. 11, the direction of the forceps channel 17 or the direction in which the medical instrument 171 emerges is indicated by an arrow-shaped directional mark. According to the present embodiment as described above, by the image processing based on the direction indicator 44 imaged by the CMOS image sensor 311, even when the camera shaft 32 rotates within the catheter 10, misrecognition of the direction of the image imaged by the CMOS image sensor 311 can be prevented.

The present invention has been described above based on the embodiments. It should be understood by those skilled in the art that the embodiments are examples, that various modifications are possible in the combination of components and processing operations, and that such modifications are also within the scope of the present invention.

Note that the functional configuration of each device described in the embodiments can be implemented by hardware resources, software resources, or cooperation of hardware resources and software resources. Processors, ROMs, RAMs, and other LSIs can be used as the hardware resources. Programs such as operating systems and applications can be used as the software resources.

### REFERENCE SIGNS LIST

10 Catheter
13 Camera channel
17 Forceps channel
23 Camera port
27 Forceps port
30 Camera
31 Camera head
32 Camera shaft
34 Distal end cover
37 Shape memory alloy tube
41 Image acquisition unit
42 Camera direction detecting unit
43 Image processing unit
44 Direction indicator
53 Camera position switching unit
100 Cholangioscope
171 Medical instrument
311 CMOS image sensor
371 Cut
381 Lead wire
382 Optical fiber

1. An endoscopic imaging device comprising:
a camera shaft configured to be inserted into a catheter and formed in a curved shape, the catheter being configured to be inserted into a body and having a curved shape; and
an imaging element provided at a distal end portion of the camera shaft.

2. The endoscopic imaging device according to claim 1, wherein curvature of the camera shaft is less than curvature of the catheter.

3. The endoscopic imaging device according to claim 1, wherein the camera shaft is formed into a curved shape by using a shape memory alloy.

4. The endoscopic imaging device according to claim 3, wherein
the shape memory alloy is not provided at the distal end portion of the camera shaft, and
the distal end portion is provided with a distal end cover covering an outer periphery of the imaging element.

5. The endoscopic imaging device according to claim 3, wherein the camera shaft is provided with a shape memory alloy tube made of the shape memory alloy, and a spiral cut is provided at least in a portion proximate to a distal end of the shape memory alloy tube.

6. The endoscopic imaging device according to claim 5, wherein the spiral cut includes a portion where a pitch of the spiral cut decreases as in a direction from a proximal end of the shape memory alloy tube to a distal end of the shape memory alloy tube.

7. An endoscope comprising:
a catheter configured to be inserted into a body and having a curved shape;
a camera shaft configured to be inserted into the catheter and formed in a curved shape; and
an imaging element provided at a distal end portion of the camera shaft.

8. The endoscope according to claim 7, wherein
the catheter further includes
an instrument channel into which a medical instrument is insertable, and
a camera channel provided in an outer side portion of the catheter having the curved shape outer than the instrument channel and into which the camera shaft is insertable.

9. The endoscope according to claim 7, further comprising a direction indicator provided at a distal end portion of the catheter and configured to be imaged by the imaging element.

10. The endoscope according to claim 7, wherein
in the catheter, a cross-sectional shape of at least a part of the camera channel into which the camera shaft is insertable and a cross-sectional shape of at least a part of the camera shaft are non-circular and substantially similar.

11. An endoscope comprising:
a catheter configured to be inserted into a body;
a camera shaft configured to be inserted into the catheter;
an imaging element provided at a distal end portion of the camera shaft; and
a direction indicator located at a distal end portion of the catheter and configured to be imaged by the imaging element.

12. The endoscope according to claim 11, wherein
the catheter further includes a camera channel into which the camera shaft is insertable and an instrument channel into which a medical instrument is insertable, and
the direction indicator is at least one of a distal end portion of the camera channel, a distal end portion of the instrument channel, or the medical instrument protruding from the distal end portion of the instrument channel.

13. The endoscope according to claim 11, further comprising
a camera position switching unit configured to switch a position of the imaging element relative to the distal end portion of the catheter between a first position proximate to a proximal end and a second position proximate to a distal end, wherein
the direction indicator is capable of being imaged at least by the imaging element at the first position.

14. The endoscope according to claim 11, further comprising:
a camera direction detecting unit configured to detect a direction of the imaging element relative to the catheter based on the direction indicator imaged by the imaging element; and
an image processing unit configured to process an image imaged by the imaging element according to the detected direction of the imaging element.

15. The endoscope according to claim 14, wherein the image processing unit rotates an image imaged by the imaging element, and thus the detected direction of the imaging element matches a predetermined direction.

16. The endoscope according to claim 15, wherein the image processing unit cuts out the image that is a target of rotation, around the center of the rotation.

17. The endoscope according to claim 14, wherein the image processing unit displays a direction of the direction indicator imaged by the imaging element in an image imaged by the imaging element.

18. An endoscope comprising:
a catheter configured to be inserted into a body;
a camera shaft configured to be inserted into the catheter; and
an imaging element provided at a distal end portion of the camera shaft, wherein
in the catheter, a cross-sectional shape of at least a part of the camera channel into which the camera shaft is insertable and a cross-sectional shape of at least a part of the camera shaft are non-circular and substantially similar.

19. The endoscope according to claim 18, wherein a cross-sectional shape of at least a part of the camera channel and a cross-sectional shape of at least a part of the camera shaft are elliptical.

20. The endoscope according to claim 18, wherein a cross-sectional shape of at least a part of the camera channel and a cross-sectional shape of at least a part of the camera shaft are polygonal.
